(19) United States
(12) Patent Application Publication
(10) Pub. No.: US 2022/0265983 A1
(43) Pub. Date: Aug. 25, 2022

(54) CATHETER SYSTEM WITH SUBCUTANEOUS, IMPLANTABLE PORT AND ULTRASOUND-GUIDED PLACEMENT METHOD

(71) Applicant: Maho Med Tech, LLC, (US)

(72) Inventor: Shawn Schumacher, Corvallis, OR (US)

(21) Appl. No.: 17/518,815

(22) Filed: Nov. 4, 2021

Related U.S. Application Data
(60) Provisional application No. 63/200,204, filed on Feb. 21, 2021.

(51) Int. Cl. A61M 39/02; A61M 5/158; A61M 25/01; A61M 19/00

(52) U.S. Cl. CPC A61M 39/0208; A61M 5/158; A61M 25/0108; A61M 19/00; A61M 2025/0166; A61M 2005/1587

(57) ABSTRACT

A catheter system includes a medication dispenser located external to a patient. A subcutaneous port displaced internal to the patient and receives a quantity of medication. The port can be filled from the medication dispenser using a Huber needle, or some other suitable needle, fluidically connected to the medication dispenser by tubing. The catheter can be placed in proximity to a patient's nervous system using ultrasound imaging guidance. A method of administering a nerve block to a patient includes the steps of placing a subcutaneous port using ultrasound imaging for guidance and administering medication via a catheter connected to the port.

### CROSS-REFERENCE TO RELATED APPLICATION

This application is related to and claims priority in U.S. Provisional Patent Application Ser. No. 63/200,204, filed Feb. 21, 2021, which is incorporated herein by reference in its entirety.

### BACKGROUND OF THE INVENTION

### 1. Field of the Invention

The present invention relates generally to catheters, and in particular to a catheter system with an implantable port for administering anesthetics and other localized pharmacologicals. A port placement method utilizes ultrasound imaging for placement in proximity to patients' nerves and nerve centers.

### 2. Description of the Related Art

Various medical procedures involve the administration of pharmacologicals for achieving favorable outcomes. For example, anesthesiology typically involves anesthetizing patients during surgery and other medical and dental procedures. General anesthetics render patients unconscious for limited, predetermined periods of time, during which medical procedures, e.g., surgeries, are performed. Local anesthetics are commonly used for anesthetizing specific areas of patients, e.g., for dental procedures, surgeries performed on extremities, etc.

The field of palliative care involves treating patients who have been diagnosed with serious illnesses. Palliative care objectives include improving patient quality of life and minimizing disruption for caregivers, e.g., medical professionals and family members. Palliative care is a growing field in medicine. Population demographics in the United States, including an aging population, are likely to contribute to more palliative care cases and greater anticipated demand for adequate pain relief. End-of-life patients are often treated by hospice care medical service providers. Patients receiving hospice care often require medications for chronic pain.

Opioids represent a significant class of pain control drugs and are commonly prescribed for and administered to patients dealing with chronic pain, including hospice care patients. However, opioid-based pharmaceuticals have multiple disadvantages. Patient addiction and opioid dependency are significant concerns. Expense and stringent regulatory (e.g., FDA) control are additional factors. Moreover, patients can develop tolerances, which can necessitate switching treatment protocols and increasing required dosages to achieve effective outcomes.

Catheters for administering medications, including anesthetics, via patients' venous circulatory systems are well-known in the art. For example, Luther U.S. Pat. No. 5,403,283 discloses a percutaneous port catheter assembly and method of use. Cal et al. U.S. Pat. No. 5,743,873 discloses methods for using catheter connectors and portals, and methods of assembly.

Anesthetics are generally most effective when administered in proximity to patients' nerves. Relatively recent improvements in ultrasound technology enable healthcare providers to more precisely visualize and locate nerves, as compared to blind catheter placement techniques used previously.

Heretofore there has not been available a catheter system and method with the advantages and features of the present invention.

### BRIEF SUMMARY OF THE INVENTION

A catheter system includes an implantable port connected to a catheter. The catheter can be placed in proximity to nerves using ultrasound imaging techniques and equipment. Optimal catheter placement is generally determined by diagnosing patients and identifying areas causing pain or otherwise requiring the application of pharmacologicals. For example, surgical sites contribute to patient pain and discomfort during the healing process. In oncology cases, chemotherapy medications are commonly administered in proximity to tumors. Various medications, including anesthetics and chemotherapy medications, can be injected or pumped into the implantable port. Huber needles are specifically designed for administering medications to the implantable ports.

A medication delivery method according to the present invention includes the steps of placing an implantable port, extending a catheter from the port to an affected area requiring treatment, and cycling the medication administration as necessary to achieve a favorable outcome, such as healing or alleviating pain and discomfort.

### BRIEF DESCRIPTION OF THE DRAWINGS

FIG. 1 a cross-sectional view of a catheter system embodying an aspect of the present invention, shown receiving a medication for transfusion in a patient.

FIG. 2 is a flowchart showing a pain management treatment protocol method embodying an aspect of the present invention.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Said terminology will include the words specifically mentioned, derivatives thereof and words of similar meaning.

### II. Catheter System 2

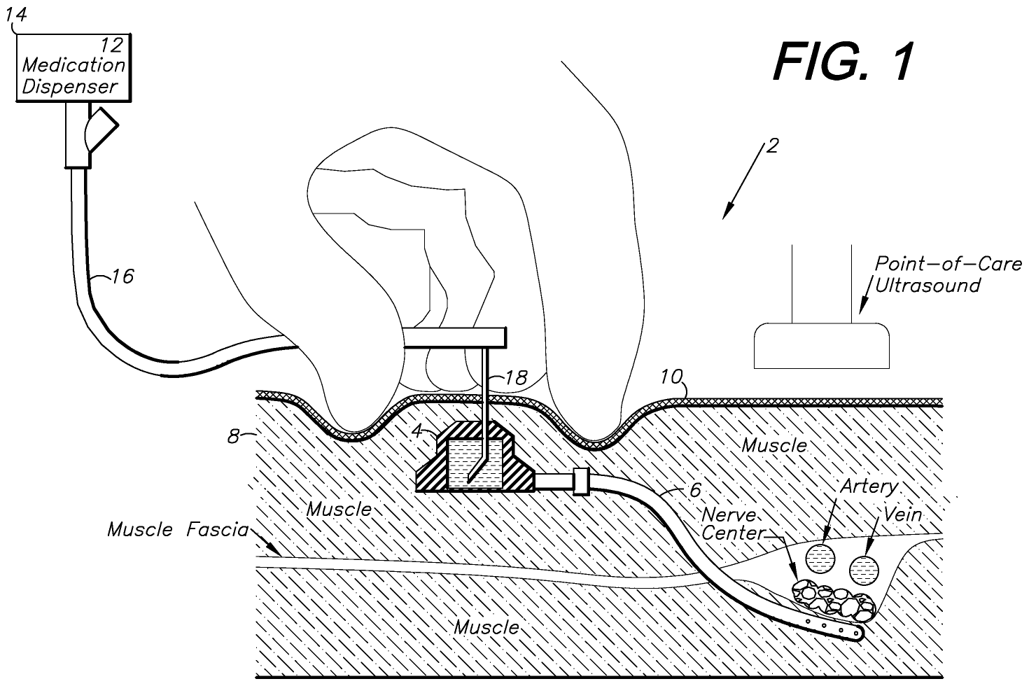

A catheter system 2 embodying an aspect of the present invention is shown in FIG. 1 and generally includes an implantable port 4 connected to a catheter 6. The port 4 is preferably placed within the patient's dermis 8, below the epidermis 10. The port 4 is preferably configured for receiving a quantity of a liquid pharmacological, such as an anesthetic, a chemotherapy medication, etc. A catheter 12 is fluidically connected to the port 6 and terminates in proximity to portions of the patient's nervous system to be anesthetized. Nerves and nerve centers can be located using an ultrasound imaging procedure.

A medication dispenser 12 can comprise a syringe 14 connected to tubing 16 terminating at a needle 18. Huber and other suitable needles can be used for injecting medications into the subcutaneous port 4. Various other medication dispensing devices can be used with the catheter system 2 of the present invention. For example, in lieu of a manually-operated syringe 14, a motorized pump can be provided. Moreover, the medication dispenser 12 can be automated with a programmable microprocessor for cycling the operation of a motorized pump to dispense medication at predetermined intervals consistent with a predetermined treatment protocol. Moreover, various medications can be selectively administered, including, without limitation, anesthetics, chemotherapy medications, growth factors, antiseptics, etc.

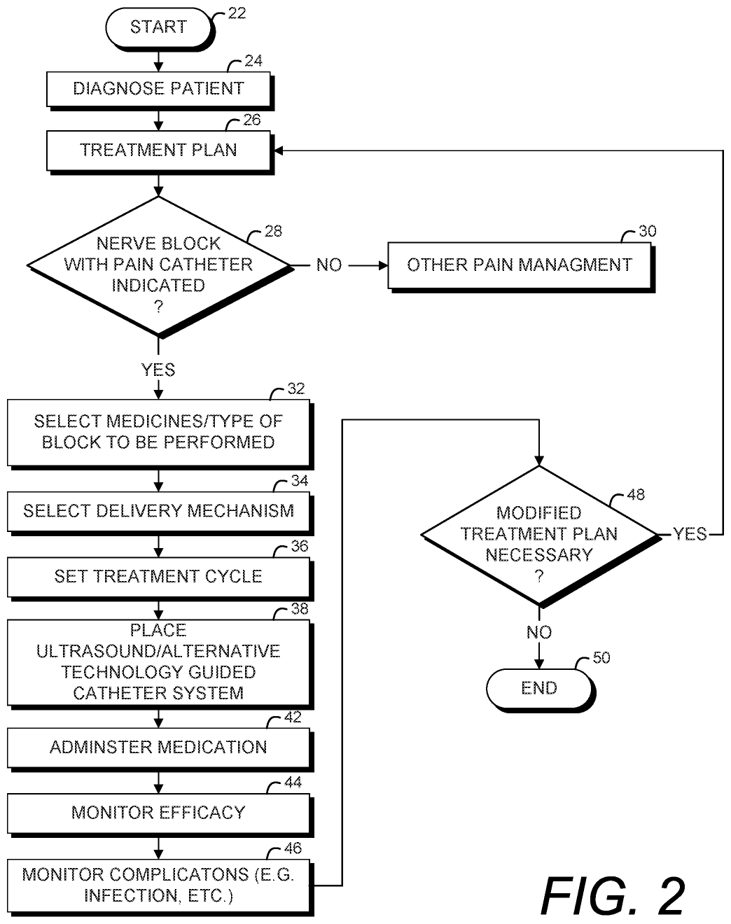

FIG. 2 is a flowchart showing an example of a medication administration method embodying an aspect of the present invention. From a start 22, the method includes the steps of diagnosing the patient at 24 and devising a treatment plan at 26. At decision box 28, a procedure including a nerve block with a pain catheter can be considered. If negative (“NO”), other pain management can be chosen at 30. If positive (“YES”), the protocol continues to selection of medicines and type of block to be performed at 32 and selection of a delivery mechanism at 34. The treatment cycle (e.g., intermittent, continuous, etc.) is set at 36. Step 38 involves placing an ultrasound/alternative technology guided catheter system.

Medication is administered at 42 and its efficacy is monitored at 44. Complications are monitored at 46 and can include, for example, infection, excessive fluid accumulation, etc. If a modified treatment plan is deemed necessary (“Yes” path from decision box 48), the protocol loops back to the treatment plan step 26. If “No,” the treatment terminates at 50.

### III. CONCLUSION

The catheter system 2 and method of the present invention can be adapted to accommodate a variety of medical conditions and treatment protocols. For example, antiseptics for infection control and growth factors for promoting reepithelialization can be introduced to a wound site.

It is to be understood that while certain embodiments and/or aspects of the invention have been shown and described, the invention is not limited thereto and encompasses various other embodiments and aspects.

1.-9. (canceled)

10. A catheter system for administering nerve block medication to a patient, which system includes:
a medication dispenser external to the patient;
a subcutaneous medication port;
a needle configured for injecting medication into said port;
tubing connected to said medication dispenser and said needle;
a nerve block catheter fluidically connected to said medication port and terminating internally within the patient in proximity to a respective patient nerve center;
a control system configured for selectively injecting medication to said catheter;
said control system configured for injecting medication in predetermined dosages;
said control system configured for injecting medication at predetermined time intervals;
an ultrasound imaging system configured for placing said catheter;
said ultrasound imaging system configured for imaging patients' neurology;
said ultrasound imaging system configured for imaging said catheter and patient nerve centers in real time;
said ultrasound imaging system configured for use in guiding said catheter for medication discharge in proximity to the patient nerve center at a location remote from said implantable port; and
said catheter system configured for administering nerve block medications.

11. A method of administering medication to a patient with ultrasound guidance, which includes the steps of:
providing a medication dispenser external to the patient;
placing a medication port subcutaneously;
injecting medication into said port;
connecting a catheter to said medication port; and
placing a discharge of said catheter internally in the patient.

12. The method according to claim 11, which includes the additional step of placing said catheter discharge in proximity to a nerve center of the patient.

13. The method according to claim 12, which includes the additional step of providing a control system configured for selectively injecting medication to said catheter.

14. The method according to claim 13, which includes the additional step of configuring said control system for injecting medication in predetermined dosages.

15. The method according to claim 14, which includes the additional step of configuring said control system for injecting medication at predetermined time intervals.

16. The method according to claim 11, which includes the additional step of providing an ultrasound imaging system configured for placing said catheter.

17. The method according to claim 16, which includes the additional step of configuring said ultrasound imaging system for imaging patients' neurology.

18. The method according to claim 17, which includes the additional steps of:
configuring said ultrasound imaging system for imaging said catheter and said patient nerve centers in real time; and
configuring said ultrasound imaging system for guiding said catheter for medication discharge in proximity to a patient nerve center.

19. The method according to claim 11, which includes the additional step of configuring said catheter system for administering nerve block medications.

20. The method according to claim 11, which includes the additional steps of:
monitoring said patient for adverse reactions to said medication; and
signaling an adverse reaction.
